(19) United States
(12) Patent Application Publication
(10) Pub. No.: US 2010/0312203 A1
(43) Pub. Date: Dec. 9, 2010

(54) Tear Away Fluid Collection Container

(75) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(73) Assignee: COLORADO CATHETER COMPANY, INC., Colorado Springs, CO (US)

(21) Appl. No.: 12/793,971

(22) Filed: Jun. 4, 2010

Related U.S. Application Data
(60) Provisional application No. 61/184,245, filed on Jun. 4, 2009.
(60) Provisional application No. 61/184,251, filed on Jun. 4, 2009.
(60) Provisional application No. 61/184,255, filed on Jun. 4, 2009.
(60) Provisional application No. 61/184,260, filed on Jun. 4, 2009.

(51) Int. Cl. A61M 27/00; B32B 38/04

(52) U.S. Cl. 604/322; 604/328; 216/33

(57) ABSTRACT

Novel catheter technology enabling use by individuals having limited dexterity are disclosed. The improvements include an apparatus and methods for emptying fluid from a fluid collection bag, a flow indicator on the fluid collection bag, and a convenient packaging for a catheterization kit containing these components. The apparatus and methods for emptying fluid from a fluid collection bag employ a drainage system including a plurality of tear off portions that expose drain tubes having different flow rates. Further, a flow indicator may be provided in the inlet port of the fluid collection bag. An attaching member is coupled to the fluid collection bag, enabling the fluid collection bag to be hung from a frame of a chair that a patient or user is sitting in.

This U.S. Utility Patent Application claims priority to U.S. Provisional Patent Application Ser. No. 61/184,245, filed Jun. 4, 2009; and to U.S. Provisional Patent Application Ser. No. 61/184,251, filed Jun. 4, 2009; and to U.S. Provisional Patent Application Ser. No. 61/184,255, filed Jun. 4, 2009; and to U.S. Provisional Patent Application Ser. No. 61/184,260, filed Jun. 4, 2009, the contents of all of which are hereby incorporated by reference in their entirety into this disclosure.

### BACKGROUND OF THE INVENTION

1. Field of the Invention

The present invention relates to fluid collection bags. More specifically, the present invention relates to catheter bags having improved features enabling usage by a person with limited dexterity.

2. Background of the Invention

Intermittent catheterization of an individual's urinary bladder is a common practice today for many persons who are in a hospital setting, a nursing home, doctor's office, rehabilitation facility or at home. For instance, a patient is sometimes catheterized to treat such conditions as urinary retention, the inability to evacuate urine, or for the purpose of obtaining a sterile urine specimen from a patient in a doctor's office or at home.

The need for intermittent catheterization of an individual is preferred to long term use of indwelling catheters, because of decreased infections, urethral damage, bladder damage, and greater personal mobility. Long term use of an indwelling catheter is also a risk factor for bladder cancer. This is often the case for persons having a neurogenic urinary condition (neurogenic bladder), such as in a spinal cord injury, multiple sclerosis, stroke, or brain injury. Conditions that interfere with the individual's ability to voluntarily void the bladder may also arise post-surgically or as a result of benign prostatic hypertrophy, prostate cancer or diabetes mellitus. Many of the affected individuals are capable of, and would prefer to perform self-catheterization. For many, the level of risk and discomfort of repeated catheterizations carried out over the course of a day (at 3-6 hour intervals, for example) are offset by the accompanying convenience, privacy or self-reliance that is achieved. Some of the major difficulties that arise in self-catheterization are the lack of satisfactory catheterization kits, the problem of maintaining the required level of sanitation during the procedure, and the difficulty of sometimes performing the procedure under conditions of restricted space and privacy.

In assisted, or non self-catheterizations, it is common practice in hospitals to employ a catheterization tray with an “open” sterile catheterization procedure, which typically includes a sterile drape, gloves, a conventional catheter, antiseptic solution, swabs, lubricant, forceps, underpad and a urine collection container. “Open” refers to the fact that the catheter is not covered or protected by a sheath or bag. In contrast, “closed” catheter systems are sterile and covered with a sheath or bag that prevents contamination. Assisted catheterization is usually performed by a healthcare professional with the patient in a supine position. Maintaining a sterile field with an open sterile catheter system can still be a problem due to the multiple steps and an unprotected catheter.

Many individuals with spinal cord injuries or other neurological diseases routinely perform self-intermittent catheterization several times a day using conventional bare or uncovered catheters and “clean technique.” Clean technique means that the urethral area is initially wiped with a moist soapy washcloth, and efforts are made to avoid contamination of the catheter during the procedure. The user's hands are not sterile and a sterile field is not present. Clean technique is used instead of sterile technique, generally, for two reasons. First, it is very difficult, if not impossible, for individuals who are performing self-catheterization to adhere strictly to sterile technique. Secondly, some studies have shown that some individuals are able to catheterize with clean technique without a statistically significant increase in urinary tract infection. Individuals reuse “cleaned” catheters after cleaning with soap and water or boiling them. Others will use non-sterile technique and develop subsequent infection of the urinary tract, causing significant morbidity and cost to the patient and society. In other cases, a bare sterile single-use catheter is used instead of clean technique. For others, the use of a closed system sterile catheter leads to a decrease in infection and an improvement of quality of life.

With most conventional self-contained sterile units where the collection bag doubles as the catheter insertion cover, the catheter is extremely difficult for the user to grasp and insert. This is particularly a problem for self-catheterization users who may also have neurological problems that limit manual dexterity. Also, with some of the available catheter kits and methods, the catheter is either not sufficiently lubricated during insertion (and thus requires the additional application of possibly non-sterile lubricant), or the catheter is too slick with lubricant and cannot effectively be grasped through an insufficiently flexible bag. As a practical matter, most individuals with limited dexterity who would prefer to self-catheterize cannot do so, and if able have difficulty maintaining the required level of sanitation using many of the existing catheterization apparatus.

Many catheterization tasks require a degree of dexterity to accomplish.

People with normal dexterity, like paraplegics, may not have use of their lower extremities, but their hands are normal. Some quadriplegics can have use of their upper extremities, having absolutely normal movement, like a paraplegic, except for normal hand dexterity. Thus, many tasks requiring a degree of hand dexterity are very difficult for these quadriplegics to accomplish.

Spinal cord injuries at the C5, C6, or C7 level affect the use of a person's hands and make these tasks difficult or impossible with current products. In addition, people who have had strokes, brain injuries, or multiple sclerosis may also require catheterization but have limited dexterity. The current catheter market does not currently support the needs of these people.

For instance, if someone uses a catheter to drain their bladder, the catheter bag will be filled with urine. Ideally, the urine should be drained from the bag into the toilet and then the bag should be thrown away, so that the urine isn't all thrown away with the bag. Hopefully, one can empty the bag quickly so that they don't have to wait for a prolonged period of time while it's draining into the toilet. Additionally, some people need to get a urine sample from the drainage bag. Obviously for a urine sample it is not ideal for fluid from the bag to be rushing out as it would if the bag was being emptied to throw away. It should be a slower, controlled process to fill up, for instance, a sample cup.

Moreover, users often want to be able to carry multiple catheterization kits with them to last them a full day. However, current models of kits have big packages due to all of the kit materials being together. These users want a smaller kit that can separated and stored in small compartments. Alternatively, when putting multiple kits even into a larger bag, a user may need to separate parts in order to fit all of the kits but desires to keep all the kits sealed and sterilized. Many users want their catheters in their fanny pack with the other part of kit in their chair. Users need more options and more variations of how to take things with them. Other users may just desire the full kit together. Manufacturers want a product that can be sold to each of these types of customers.

Therefore, what is needed is a catheterization bag for those individuals who cannot use any of the materials on the market today because of limited dexterity. The bag should allow a person with limited dexterity to empty the bag or collect a urine sample.

### SUMMARY OF THE INVENTION

The present invention provides several improvements to existing catheter technology enabling use by individuals having limited dexterity. The improvements include an apparatus and methods for emptying fluid from a fluid collection bag, a flow indicator on the fluid collection bag, an attaching member coupled to the collection bag, and a convenient packaging for a catheterization kit containing these components. The apparatus and methods for emptying fluid from a fluid collection bag employ a drainage system including a plurality of tear off portions that expose drain tubes having different diameters. The different diameters provide different flow rates for draining the fluid collection bag. Finger-sized holes on the tear away portions enable one with limited dexterity to drain the bags using only a pull of a finger. Further, a flow rate indicator is provided in the form of a flexible bubble. In embodiments utilizing the flow indicator, fluids pass through the bubble before entering a reservoir of the fluid collection bag. As the fluid passes through, the bubble expands, being made of a flexible material. Moreover, an attaching member is coupled to the fluid collection bag, enabling the fluid collection bag to be hung from a frame or cushion handle of a chair that a patient or user is sitting in. The attaching member is welded into the bag, or can clamp onto the bag. The attaching member is coupled to a cylindrical support member that additionally adds structural support to an inlet port that the catheter couples to.

In one exemplary embodiment, the present invention is a fluid collection bag in fluid communication with a catheter. The fluid collection bag includes a reservoir; an inlet tube in fluid communication with the catheter; a drain tube including a large-diameter tube in fluid communication with a small diameter tube, wherein an open end of the large-diameter tube enters the reservoir via a corner of the reservoir, and wherein a closed end of the small diameter tube is sealed at a corner of the fluid collection bag; a first tearable portion which, when detached from the fluid collection bag, exposes the closed end of the small-diameter tube to an external environment, allowing a fluid to flow from the reservoir at a slow rate; and a second tearable portion which, when detached from the fluid collection bag, exposes the large-diameter tube, allowing a fluid to flow from the reservoir at a relatively faster rate. The first and second tearable portions each include a finger-sized hole enabling a user to detach the tearable portions using a finger.

In another exemplary embodiment, the present invention is a package for a catheterization kit. The package includes a first enclosed portion containing a catheter and a fluid collection bag, and having a first flap; a second enclosed portion containing a pair of gloves, a supply of anti-septic swabs, and a gauze pad, and having a second flap, wherein the second flap is attached to the first flap along a perforated line; a finger hole on either side of the perforated line between the first and second flaps; wherein a user's fingers are placed inside the finger holes, then pulled apart to separate the first enclosed portion from the second enclosed portion along the perforated line. The fluid collection bag includes a drain tube having two tube portions, each having a different diameter, and a tearable portion for each tube portion, such that a first tearable portion is detached to drain the fluid collection bag via a small-diameter tube portion, and a second tearable portion is detached to drain the fluid collection bag via a large-diameter tube portion, each tearable portion including a finger-sized hole to detach the tearable portion from the fluid collection bag.

In yet another exemplary embodiment, the present invention is a method of manufacturing a fluid collection bag for a catheter. The method includes welding a perimeter of two substantially rectangular sheets together to form a reservoir, wherein an inlet tube portion provides fluid communication between the reservoir and a catheter tube, and wherein a drain tube portion provides fluid communication between the reservoir and an external environment of the reservoir, said drain tube portion including a small-diameter portion and a large diameter portion; sealing an end of the small-diameter portion; etching a first tear line at a corner of the two rectangular sheets traversing the small-diameter portion of the drain tube to create a first tear-away portion; etching a second tear line at the same corner of the of the two rectangular sheets traversing the large-diameter portion of the drain tube to create a second tear-away portion; and cutting a finger-sized hole in each tear-away portion to enable a user to detach the tear-away portion by pulling with a finger.

### BRIEF DESCRIPTION OF THE DRAWINGS

FIG. 1 shows a catheter and a fluid collection bag, according to an exemplary embodiment of the present invention.

FIG. 2 shows a fluid collection bag, according to an exemplary embodiment of the present invention.

FIG. 3 shows a drain tube of a fluid collection bag, according to an exemplary embodiment of the present invention.

FIG. 4 shows tear away portions and a flow indicator on a fluid collection bag, according to an exemplary embodiment of the present invention.

FIG. 5 shows an attaching member coupled to a fluid collection bag, according to an exemplary embodiment of the present invention.

FIG. 6 shows an attaching member removably coupled to a fluid collection bag, according to an exemplary embodiment of the present invention.

FIG. 7 shows a package for a catheterization kit, according to an exemplary embodiment of the present invention.

FIG. 8 shows a method of manufacturing a fluid collection bag, according to an exemplary embodiment of the present invention.

### DETAILED DESCRIPTION OF THE INVENTION

The present invention provides several improvements to existing catheter technology enabling use by individuals having limited dexterity. The improvements include an apparatus and methods for emptying fluid from a fluid collection bag, a flow indicator on the fluid collection bag, and a convenient packaging for a catheterization kit containing these components. The apparatus and methods for emptying fluid from a fluid collection bag employ a drainage system including a plurality of tear off portions that expose drain tubes having different flow rates. Further, a flow indicator is provided in the inlet port of the fluid collection bag. As the fluid passes into the fluid collection bag, a flexible bubble expands, and contracts when the flow ceases. An attaching member is coupled to the fluid collection bag, enabling the fluid collection bag to be hung from a frame of a chair that a patient or user is sitting in.

Embodiments of the present invention include a corner or section of the fluid collection bag with a plurality of thumb or finger holes. These thumb or finger holes allow at least two portions of the fluid collection bag to be torn off, one of which provides a slow flow rate and the second providing a faster flow rate. This is achieved by providing a drain tube having at least two different diameters, each one corresponding to a flow rate. Perforated or weakened material lines between an outside finger hole and a middle hole and between the middle hole and an inside hole allow the tear off portions to be detached relatively easily but not accidentally. These perforations are created during the manufacturing of the fluid collection bag. The manufacturing involves sealing outer edges of two plastic sheets wherein the unsealed portion in the middle creates a reservoir and the drain port. Further, an inlet port is provided, the inlet port enabling a catheter to be in fluid communication with the reservoir. The reservoir collects fluid from the catheter and stores the fluid until it is emptied. When the tear away portions of the fluid collection bag are detached, fluid from the reservoir flows out via the drain tube into an external environment.

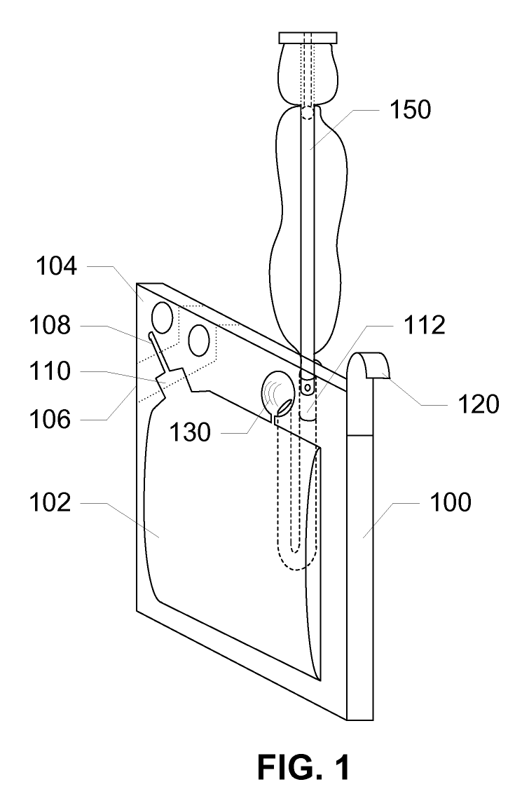

FIG. 1 shows a catheter 150 and a fluid collection bag 100, according to an exemplary embodiment of the present invention. In this embodiment, fluid collection bag 100 includes a reservoir 102, a first tearable portion 104, a second tearable portion 106, a small-diameter tube 108, a large-diameter tube 110, an inlet port 112, a flow indicator 130, and an attaching member 120. Inlet port 112 provides fluid communication between reservoir 102 and catheter 150. Catheter 150 couples to fluid collection bag 100 at inlet port 112, such that fluid flowing through catheter 150 flows into reservoir 102 of fluid collection bag 100. Fluid collection bag 100 includes first tearable portion 104, which when torn exposes small-diameter tube 108 to an external environment. Fluid collection bag 100 also includes second tearable portion 106, which when detached exposes large-diameter tube 110 to an external environment. A user may insert catheter 150 into his or her urethra. Fluid flows through catheter 150 and into reservoir 102, through inlet port 112. With urine or other fluids in fluid collection bag 100, user may tear off first tearable portion 104, enabling a slow flow from reservoir 102 through small-diameter tube 108. This may allow the user, for instance, to collect a urine sample. When the user is finished taking the sample, the user may tear off second tearable portion 106, creating a faster flow through large-diameter tube 110, in order to empty fluid collection bag 100. If the user does not need a sample but wishes to empty fluid collection bag 100, the user may tear off second tearable portion 106 without tearing off first tearable portion 104. Flow indicator 130 is, in this embodiment, in the form of a flexible bubble stamped into the plastic of fluid collection bag 100, and in fluid communication with reservoir 102. The bubble 130 expands as fluid flows through into reservoir 102, and collapses when the flow ceases. This enables a user to be aware of when the flow is complete. Finally, attaching member 120 provides a means to attach fluid collection bag 100 to, for instance, a wheelchair. This may prevent catheter 150 from being pulled out of the user's urethra during catheterization due to the weight of fluids in fluid collection bag 100.

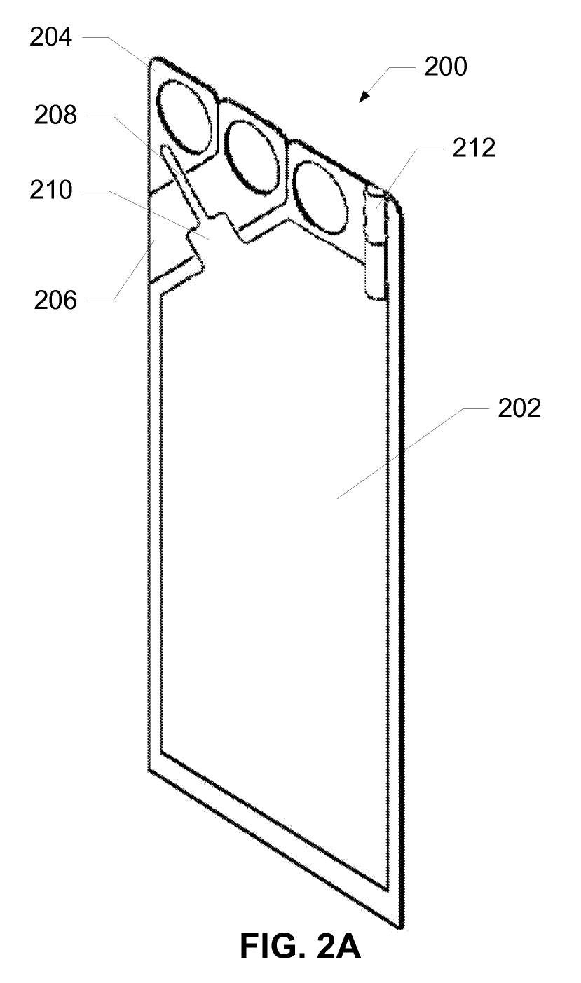

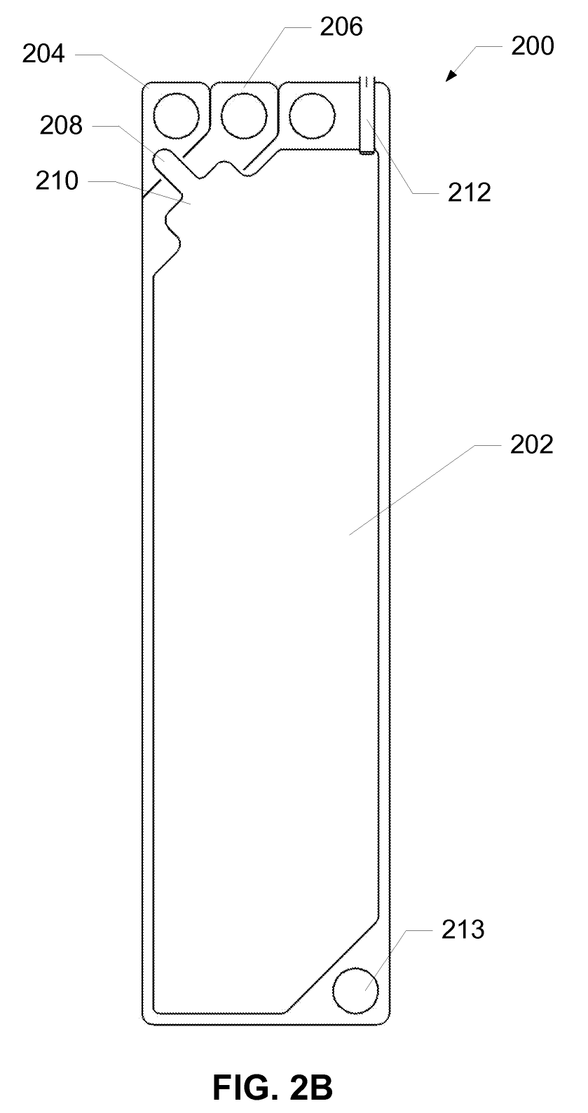

FIG. 2A shows a fluid collection bag 200, according to an exemplary embodiment of the present invention. Fluid collection bag 200 is composed of a flexible material, such as polyvinylchloride (PVC), capable of holding a fluid. Fluid collection bag 200 is stamped and welded to create a reservoir 202, a first tearable portion 204, a second tearable portion 206, a small-diameter tube 208, a large-diameter tube 210, and a fluid inlet 212. Such welding may be accomplished, for instance, by radio frequency (RF) welding. The thickness of fluid collection bag 200 is such that it remains flexible while not being easily punctured or ripped. Embodiments of fluid collection bag 200 are constructed of PVC sheets that are between 0.005 and 0.01 inches, or between 5-10 mils thick. Inlet port 212 provides a location for providing fluid communication between a catheter and reservoir 200. Inlet port 212 allows fluid to flow from the catheter into reservoir 202 of fluid collection bag 200. Reservoir 202 is a portion of fluid collection bag 200 where urine or any other fluid is collected, and is in fluid communication with inlet port 212, large-diameter tube 210, and small-diameter tube 208. Small-diameter tube 208 is a portion of fluid collection bag 200 that is in fluid communication with reservoir 202 and, when exposed to an external environment, allows for a slow flow rate of fluid from reservoir 202 to the external environment. The end of small-diameter tube 208 farthest from reservoir 202 is exposed to the external environment by tearing first tearable portion 204 from fluid collection bag 200. Small-diameter tube 208 may be used, for instance, to collect a urine sample from reservoir 202. Large-diameter tube 210 is a portion of fluid collection bag 200 that is in fluid communication with reservoir 202 and, when exposed to the external environment, allows for a faster flow of fluid from reservoir 202. The end of large-diameter tube 210 farthest from reservoir 202 is exposed to the external environment by tearing second tearable portion 206. In this embodiment, small-diameter tube 208 is in fluid communication with reservoir 202 through large-diameter tube 210. However, small-diameter tube 208 and large-diameter tube 210 may be located anywhere on fluid collection bag 200 such as on the lower left or right corner.

As mentioned herein, the present invention allows for one with limited manual dexterity to drain a fluid collection bag. For instance, pulling two holes in different directions causes the perforated line to tear and a tear-away portion to separate from the fluid collection bag. The perforated line runs from a first edge of the fluid collection bag to a second edge of the fluid collection bag, while intersecting a collection tube or hole. This opens up the collection tube or hole when the portion has been torn off. Fluid flows through the collection tube or hole from the reservoir to an external environment of the fluid collection bag. Fluid exiting the fluid collection bag may snake back and forth along an extended drain tube, helping to control and slow the process in order to drain and fill up, for instance, a urine sample cup. Further, the collection tube or hole may be stamped into the plastic of the fluid collection bag, may be a tube inserted into the corner of the fluid collection bag during manufacturing, etc. In embodiments of the invention, this tube or hole may have a decreasing diameter to further slow the flow.

FIG. 2B shows a fluid collection bag 200, according to an exemplary embodiment of the present invention. In this embodiment, fluid collection bag 200 includes a reservoir 202, a first tearable portion 204, a second tearable portion 206, a small-diameter tube 208, a large-diameter tube 210, a fluid inlet 212, and a lower finger hole 213. The addition of lower finger hole 213 allows a user to insert a finger into lower finger hole 213 in order to tip fluid collection bag 200 upside down to drain fluid collection bag 200 through large-diameter tube 210 or take a sample through small-diameter tube 208. Additionally, lower finger hole 213 may be clipped to a hole in the upper portion of fluid collection bag 200 to reduce the length of fluid collection bag 200. This may, for instance, prevent fluid collection bag 200 from dragging, etc.

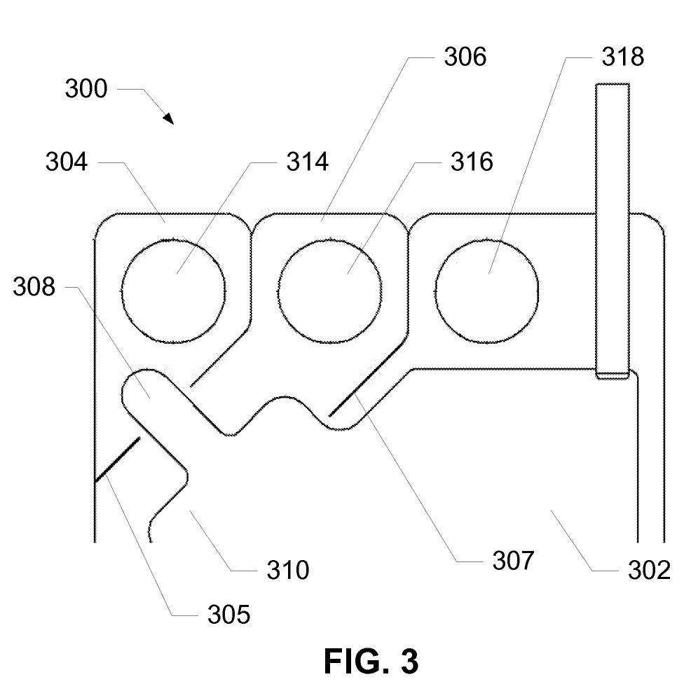

FIG. 3 shows tearable portions of a fluid collection bag 300, according to an exemplary embodiment of the present invention. According to this embodiment, a first tearable portion 304 and a second tearable portion 306 may be detached to respectively expose a small-diameter tube 308 and a large-diameter tube 310 to an external environment. First tearable portion 304 is a portion of fluid collection bag 300 that may be torn off, exposing small-diameter tube 308 to the external environment. First tearable portion 304 includes a first tear line 305, which is welded to a different degree than the rest of fluid collection bag 300, or perforated, allowing the user to more easily tear along first tear line 305. First tear line 305 extends through a portion of small-diameter tube 308. To remove first tearable portion 304, the user inserts a finger into a first hole 314 on first tearable portion 304 and a second hole 316 on second tearable portion 306. The user then pulls first hole 314 and second hole 316 in opposite directions to tear along first tear line 305. Removing first tearable portion 304 exposes small-diameter tube 308 to the external environment, creating a slow flow from reservoir 302 when reservoir 302 is tipped upside down, squeezed, etc. Second tearable portion 306 is a portion of fluid collection bag 300 that may be torn off, exposing large-diameter tube 310 to the external environment. Second tearable portion 306 includes a second tear line 307, similar to first tear line. Second tear line 307 extends through a portion of large-diameter tube 310. To remove second tearable portion 306, the user inserts a finger into second hole 316 on second tearable portion 306 and a third hole 318 on fluid collection bag 300. The user then pulls second hole 316 and third hole 318 in opposite directions to tear along second tear line 307. Removing second tearable portion 306 exposes large-diameter tube 310 to the external environment, creating a faster flow from reservoir 302 when reservoir 302 is tipped upside down, squeezed, etc. First hole 314, second hole 316, and third hole 318 may be reinforced such that tearing only occurs along the tear lines. This reinforcement may be accomplished by utilizing extra material around the hole, placing a ring of another material around the hole, etc. Alternatively, the portions may have tabs to grab instead of holes. These tab portions may be on the same corner, separate corners, or anywhere else on the collection bag.

Embodiments of the present invention include a flow indicator. The flow indicator can be a bubble that inflates when fluid is flowing and deflates such that it is flat when the flow has stopped. A catheter enters the fluid collection bag and an inlet of the flow indicator. The pressure of the flowing fluid causes the bubble to inflate. Without this pressure, the bubble deflates. In this way, a user can determine when the flow of a fluid has stopped before removing the catheter.

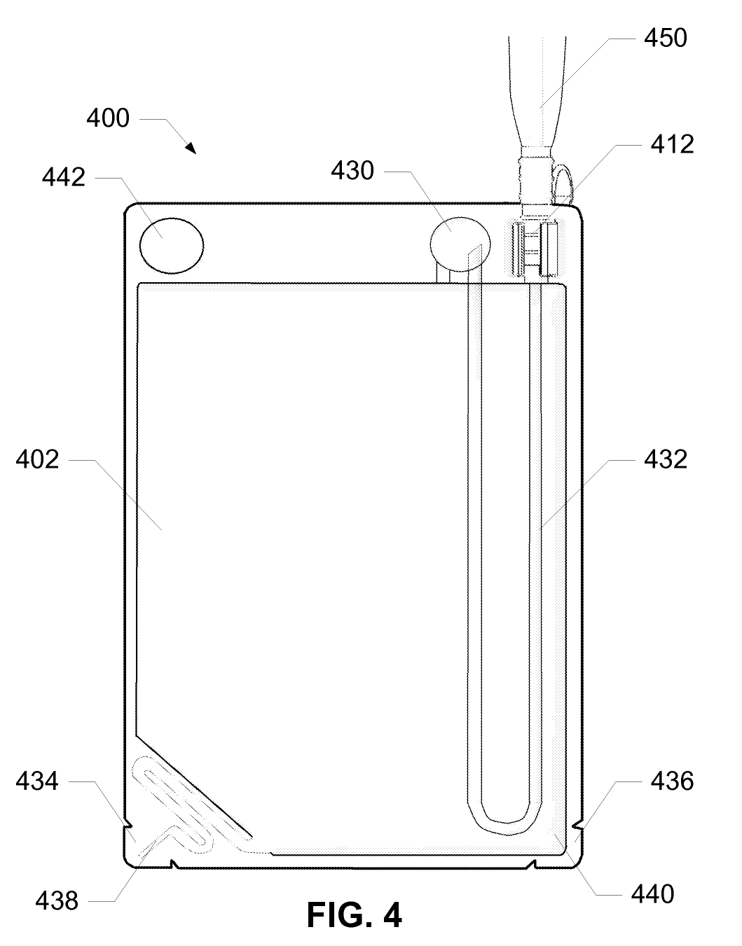

FIG. 4 shows tear away portions and a flow indicator 430 on a fluid collection bag 400, according to an exemplary embodiment of the present invention. In this embodiment, fluid collection bag 400 includes flow indicator 430, an inlet port 412, an inlet tube 432, a reservoir 402, a large-diameter hole 440, a small-diameter snaking tube 438, a first tab 434, a second tab 436, and a grip hole 442. When a catheter 450 is being used, fluid flows from catheter 450 through inlet port 412 and into inlet tube 432. Inlet tube 432 is formed of any material capable of forming a tube to direct fluids, such as PVC. Inlet tube 432 contains a U-shaped portion which is located within reservoir 402, but fluid within inlet tube 432 is not directly in fluid communication with reservoir 402. For instance, inlet tube 432 can be a separate tube that is sandwiched between two plastic sheets that form fluid collection bag 400, having an opening into flow indicator 430. Fluid from inlet tube 432 exits into flow indicator 430. Flow indicator 430 is formed of a flexible material which tends to collapse when a fluid is not flowing through it, but easily inflates as the fluid flows through. This material may be, for instance, PVC. Fluid from catheter 450 flows through flow indicator 430 and into reservoir 402. When fluid flows through inlet tube 432 and into flow indicator 430, flow indicator 430 expands. When fluid stops flowing through flow indicator 430, flow indicator 430 contracts. Thus, flow indicator 430 may be used to determine whether or not fluid is flowing into reservoir 402 when catheter 450 is being used. Grip hole 442 may be utilized to hold fluid collection bag 400 and is designed such that a user with limited dexterity may loop a finger through grip hole 442 to hold fluid collection bag 400. In this embodiment of the invention, small-diameter snaking tube 438 and large-diameter hole 440 are positioned at lower corners of fluid collection bag 400. Similar to the tearable portions of the above figures, first tab 434 and second tab 436 may be easily torn off of fluid collection bag 400. Removing first tab 434 exposes small-diameter snaking tube 438 to an external environment. This allows fluid to slowly flow from reservoir 402 to the external environment. Small-diameter snaking tube 438 allows the user to see the fluid as it flows through small-diameter snaking tube 438. This may be useful, for instance, when collecting a sample from reservoir 402. Removing second tab 436 exposes large-diameter hole 440 to the external environment. This allows fluid to quickly flow from reservoir 402 to the external environment. This may be useful, for instance, when emptying reservoir 402. While this embodiment shows first tab 434, second tab 436, small-diameter snaking portion 438, and large-diameter hole 440 in the bottom corners of fluid collection bag 400, these features may located anywhere on fluid collection bag 400.

In exemplary embodiment of the present invention, the flow indicator is a bubble that is stamped into the plastic of the catheter bag. The bubble is ideally transparent and made of a non-rigid material such that it expands when fluid is flowing through the catheter and into the flow indicator. In alternative embodiments of the present invention, the flow indicator is not stamped into the plastic but is an external piece. This external piece may itself be a bubble. The external piece may be transparent such that one can see it filling up. Additionally, the external piece may have a ball internal to it in order to determine a flow. This ball may be displaced from an original location by the flow of fluid and return to an original position when the flow has stopped. The external piece may be located anywhere along the flow of fluid between a proximal end and the reservoir such that a user is able to observe the expansion and contraction of the indicator.

The present invention further provides an apparatus and methods for attaching a reservoir bag to a seat in which a user is seated during catheterization. An attachment member, for instance a hook, is coupled with the fluid collection bag near a junction with a catheter. The attachment releasably attaches the fluid collection bag to the seat so that the seat supports the weight of the fluid collection bag. The fluid collection bag hangs from the attaching member allowing fluid to flow downward from the catheter into the fluid collection bag. Using the fluid collection bag in this manner relieves the user from supporting the weight of the fluid collection bag during catheterization.

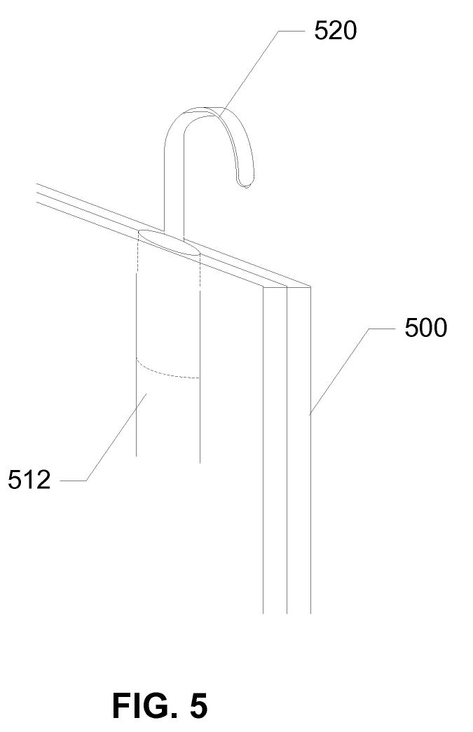

FIG. 5 shows an attaching member 520 coupled to a fluid collection bag 500, according to an exemplary embodiment of the present invention. In this embodiment, a portion of attaching member 520, or hook, is sandwiched between the two plastic sheets that form fluid collection bag 500. The lower portion of hook 520 is formed around an inlet port 512. During manufacture, hook 520 is coupled to inlet port 512, and the outer edges of the two plastic sheets can be welded to form fluid collection bag 500. Hook 520 includes a lower portion that wraps around inlet port 512, providing structural support and rigidity to inlet port 512, thereby avoiding a bend or blockage of the flow into a reservoir of fluid collection bag 500. Alternatively, hook 520 and inlet port 512 may be a single molded piece sandwiched within fluid collection bag 500.

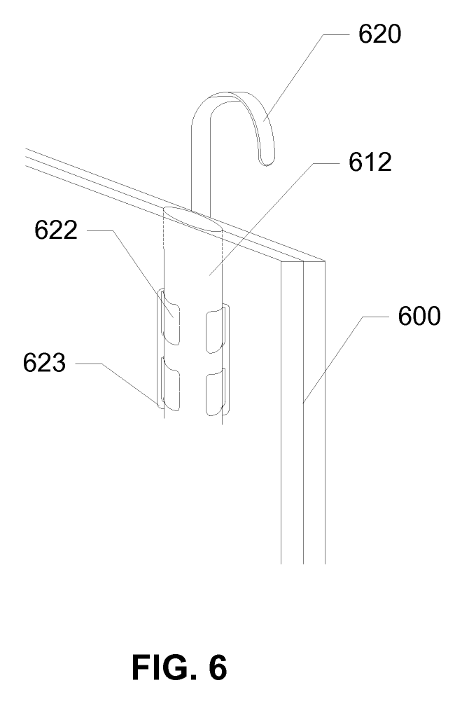

FIG. 6 shows a hook attachment 620 removably coupled to a fluid collection bag 600, according to an exemplary embodiment of the present invention. Fluid collection bag 600 includes inlet port 612, and slits around inlet port 612 for attachment clamps 622. Attachment clamps 622 are part of a lower portion of hook 620. In this embodiment, hook 620 is external to fluid collection bag 600, and clamps 622 traverse the slots and grip inlet port 612. Clamps 622 are sized to snugly match the diameter of inlet port 612. In this manner, clamps 622 fixedly but releasably snap onto inlet port 612. At the same time, the slits prevent hook 620 and clamps 622 from moving along inlet port 612 relative to fluid collection bag 600. During catheterization, a user clamps attaching member 620 to fluid collection bag 600. Once clamped, the user attaches fluid collection bag 600 to a seat in which the user is sitting. Before catheterization, while the fluid collection bag 600 is still empty, attaching member 620 does not come loose despite jostling the catheter into position within inlet port 612. During catheterization, the user does not have to adjust to the increasing weight of fluid collection bag 600 because the weight is supported completely by the seat. After catheterization, the attaching member 620 is released from the seat where it is taken to another location for emptying.

In other exemplary embodiments, the hook is longer and closer to the bag in what resembles a bobby pin. This embodiment rids any tendency to easily come off a strap or arm when the fluid collection bag is empty. Preparing for catheterization involves manipulating the catheter and, when there is no weight in the fluid collection bag, the hook may be detached. This embodiment positions the hook closer to the bag so it is a tighter fit. In other embodiments, a hook is utilized to hang from a front strap or bar of a wheelchair. For upholstery, a hook-and-loop fastener may be included.

In exemplary embodiments of the present invention, other elements may be incorporated into the catheter, as described in the '075 patent. For instance, near the proximal end of the catheter is a membrane or diaphragm situated between the interior space of the catheter and the guide/reservoir portion. The membrane holds lubricant inside a reservoir until it is needed for lubricating the tip and the outside surface of the catheter. The membrane may be made of, for example, silicon. In some exemplary embodiments, the catheter is positioned in a guide so the attachment locations of the circular sheath keep the catheter from falling into the sheath.

The present invention also provides an apparatus and methods for packaging catheterization kits. The packaging divides the catheter and bag from the rest of the kit, each section being individually sealed such that the two sections can be pulled apart while remaining sterile. The packaging includes thumb or finger holes such that the sections can easily be separated and opened, even by one with limited or absent dexterity.

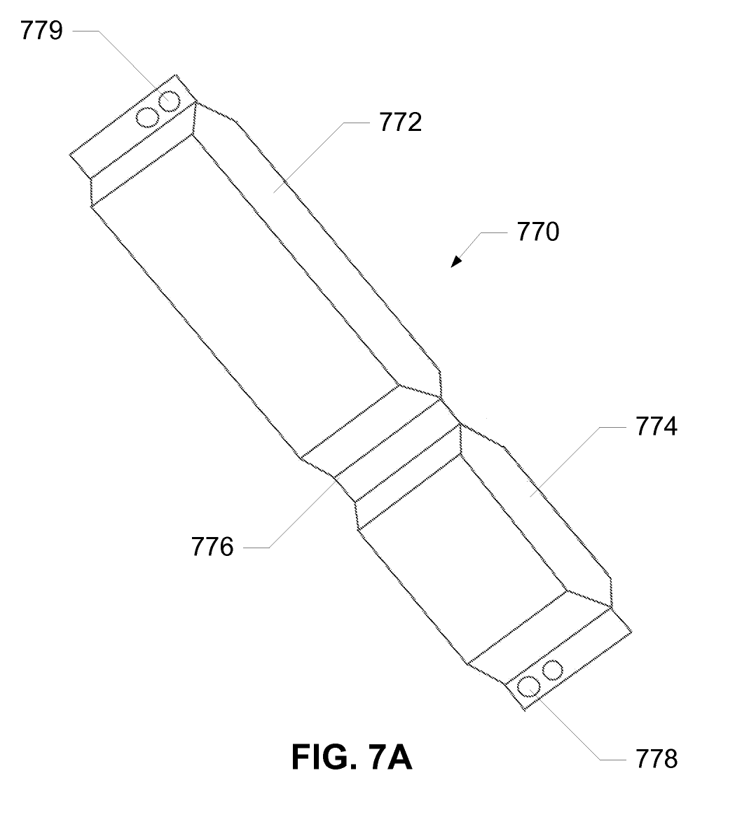

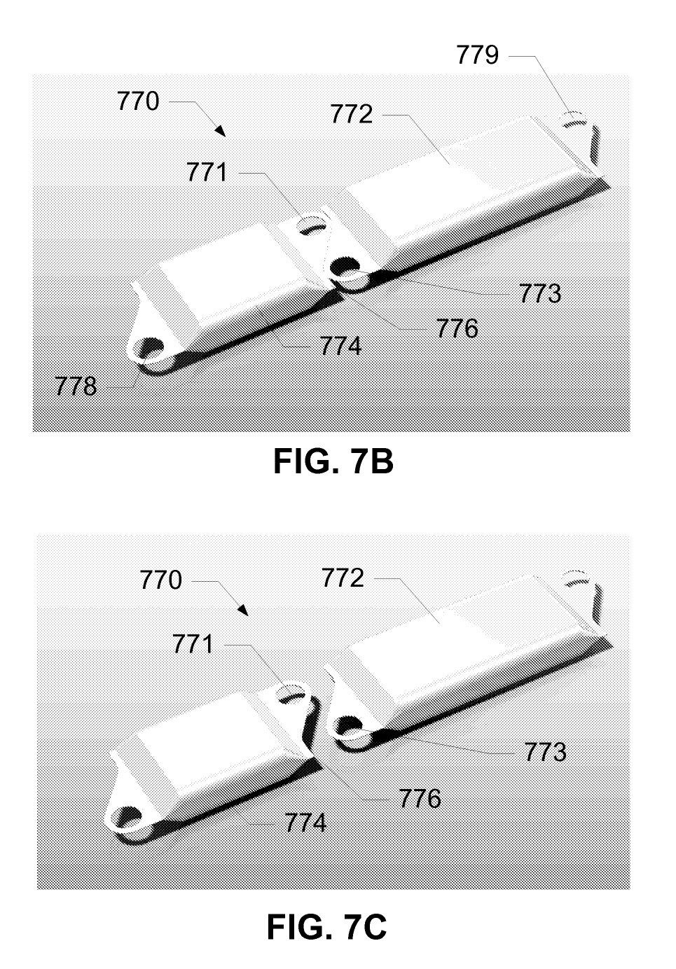

FIG. 7A shows a package 770 for a catheterization kit, according to an exemplary embodiment of the present invention. According to this embodiment, package 770 is divided into two parts, a catheter portion 772 and an accessories portion 774. These parts are both individually sealed and packaged together, but such that the two parts can be separated along a perforation line 776 while maintaining sterility after separation. Catheter portion 772 includes a catheter and a fluid collection bag. The catheter in catheterization kit may be a catheter, such as the catheter presented the '075 patent. Accessories portion 774 includes a compartment for gloves, anti-septic swabs, a 4×4 gauze, and other items in the kit. The two parts are each sealed such that they remain sterilized when separated. The separation occurs along perforated line 776 between the two parts. Each part may have a finger hole (not shown) near perforated line 776 such that when the finger holes are pulled in opposite directions, the parts are separated along perforated line 776. Perforated line 776 allows the parts to be separated relatively easily, even by someone with limited or absent manual dexterity, without easily being separated accidentally. This allows the two parts to be separated even by one with limited or absent manual dexterity. Therefore, for instance, a user tears catheterization portion 772 off and puts one section in one part of his or her backpack and the other section in another location to maximize storage and space management. Alternatively, each part may have a protruding flap near the perforated line such that the flaps can be pulled apart to separate the parts at the perforated line.

Additionally, on each part of package 770, there is another finger hole mechanism 778, 779 to assist in the opening of package 770. Each part of package 770 has a finger hole or pair of finger holes 778, 779 that can be pulled in opposite directions to tear a perforation in package 770. These finger holes 778, 779 allow a portion of packaging 770 to be torn off, exposing the contents of the kit. The two finger holes on each part may include the finger holes that are also used to separate the two parts. Thus, in embodiments, there are four finger holes. One hole on one end, one hole on the other end, and two holes in the middle so a users thumbs are put through the holes to tear along the perforation.

FIG. 7B shows a package 770 for a catheterization kit, according to an exemplary embodiment of the present invention. In this embodiment, package 770 includes a catheter portion 772 and an accessories portion 774. These parts are both individually sealed and packaged together, but such that the two parts can be separated along a perforation line 776. On either side of perforation line 776 is first separation hole 771 and second separation hole 773. First separation hole 771 and second separation hole 773 enable a user with limited dexterity to separate catheter portion 772 from accessories portion 774. On either end of packaging 770 are finger holes 778 and 779. The material of packaging 770 where finger holes 778, 779 are located is easily separated or already separated, such that both of finger holes 778, 779 go through a top sheet of material and a bottom sheet of material. The top and bottom sheets are sandwiched together, but may be peeled apart using finger holes 778, 779. For instance, the user puts one finger in finger hole 778 in top layer and the other finger in the same finger hole, finger hole 778, in the bottom layer and pulls the packaging material apart.

FIG. 7C shows a package 770 for a catheterization kit, according to an exemplary embodiment of the present invention. In this embodiment, package 770 from FIG. 7B is shown in a separated state. A user has inserted a finger in first separation hole 771 and a finger in second separation hole 773 and separated a catheter portion 772 from an accessories portion 774 along a perforation line 776.

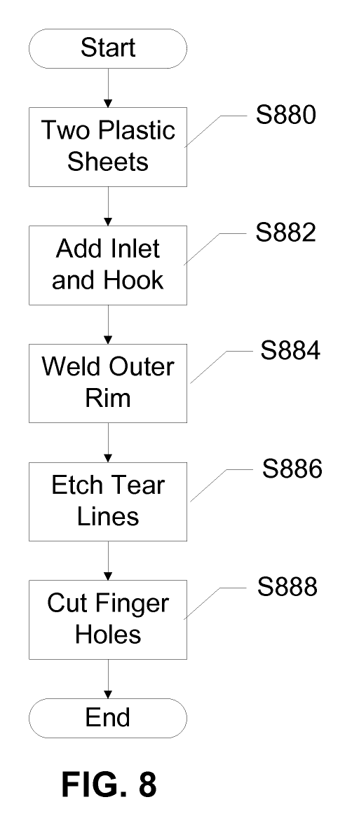

FIG. 8 shows a method of manufacturing a fluid collection bag, according to an exemplary embodiment of the present invention. The method includes providing two sheets S880 that form a laminate around a reservoir. The two sheets may be made of a plastic material that is between 5 mils and 10 mils in thickness, where 1 mil= 1/1000 of an inch. An inlet is placed between the plastic sheets S882. The inlet is a semi-rigid plastic tube that enables coupling with a distal end of a catheter, such as the catheter described in the '075 patent. The inlet port provides fluid communication between the reservoir and the catheter. Optionally, step S882 includes a hook coupled to the inlet portion. This is the hook welded into the fluid collection bag, as described with respect to FIG. 5. At step 884, an outer rim or edge of the plastic sheets is welded together to form a laminate or seal. The inner portion of the plastic sheets remains separated to form the reservoir connected to the inlet port. Further, welding step S884 includes welding around a drain port, such that a portion of the reservoir includes a drain port extending out toward an edge of the fluid collection bag. The drain port includes a large-diameter portion and a small-diameter portion to provide different flow rates. At step S886, tear lines are etched to create tear-away portions. This is done by etching a first tear line at a corner of the two rectangular sheets traversing the small-diameter portion of the drain tube to create a first tear-away portion, and etching a second tear line at the same corner of the of the two rectangular sheets traversing the large-diameter portion of the drain tube to create a second tear-away portion. Finally, at step S888, finger-sized holes are cut in each tear-away portion to enable a user to detach the tear-away portion by pulling with a finger.

Etching the tear lines may include etching a perforation along the tear lines. The welding can be performed via radiofrequency (RF) welding. In an alternate embodiment, etching the tear lines includes RF welding along the tear lines at a different degree than at the edges of the plastic sheets. In alternate embodiments, the drain port is not welded, and therefore provides fluid communication between the reservoir and an outer environment, wherein an end of the small-diameter portion of the drain port is sealed. The plastic sheets have been shown to be substantially rectangular, however other shapes are possible. The plastic material of the sheets may be polyvinylchloride (PVC) and is not limited to the thickness shown.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A fluid collection bag in fluid communication with a catheter, the fluid collection bag comprising:
a reservoir;
an inlet tube in fluid communication with the catheter;
a drain tube including a large-diameter tube in fluid communication with a small diameter tube, wherein an open end of the large-diameter tube enters the reservoir via a corner of the reservoir, and wherein a closed end of the small diameter tube is sealed at a corner of the fluid collection bag;
a first tearable portion which, when detached from the fluid collection bag, exposes the closed end of the small-diameter tube to an external environment, allowing a fluid to flow from the reservoir at a slow rate; and
a second tearable portion which, when detached from the fluid collection bag, exposes the large-diameter tube, allowing a fluid to flow from the reservoir at a relatively faster rate,
wherein the first and second tearable portions each include a finger-sized hole enabling a user to detach the tearable portions using a finger.

2. The fluid collection bag of claim 1, wherein the first and second tearable portions are detachable along any combination of a perforation, a guidance line, and a tear line.

3. The fluid collection bag of claim 1, wherein the first tearable portion is a corner portion at a corner of the fluid collection bag, and the second tearable portion is a larger corner portion at the same corner of the fluid collection bag.

4. The fluid collection bag of claim 1, further comprising an attaching member coupled to a top of the fluid collection bag, wherein the attaching member attaches to a seat in which a user is sitting during catheterization.

5. The fluid collection bag of claim 4, wherein the attaching member is coupled to the inlet port and provides structural support to the inlet port.

6. The fluid collection bag of claim 4, wherein the attaching member is removably clamped around the catheter coupled to the inlet port.

7. The fluid collection bag of claim 4, wherein the attaching member is welded into a material of the fluid collection bag.

8. The fluid collection bag of claim 1, further comprising a bubble stamped into a material of the fluid collection bag separate from the reservoir, the bubble including an inlet and an outlet, the inlet being in fluid communication with a catheter, and the outlet being in fluid communication with the reservoir, wherein a pressure of a fluid flowing through the bubble expands the bubble, and wherein a lack of pressure enables the bubble to deflate into a flattened state.

9. A package for a catheterization kit, comprising:
a first enclosed portion containing a catheter and a fluid collection bag, and having a first flap;
a second enclosed portion containing a pair of gloves, a supply of anti-septic swabs, and a gauze pad, and having a second flap, wherein the second flap is attached to the first flap along a perforated line;
a finger hole on either side of the perforated line between the first and second flaps;
wherein a user's fingers are placed inside the finger holes, then pulled apart to separate the first enclosed portion from the second enclosed portion along the perforated line, and
wherein the fluid collection bag includes a drain tube having two tube portions, each having a different diameter, and a tearable portion for each tube portion, such that a first tearable portion is detached to drain the fluid collection bag via a small-diameter tube portion, and a second tearable portion is detached to drain the fluid collection bag via a large-diameter tube portion, each tearable portion including a finger-sized hole to detach the tearable portion from the fluid collection bag.

10. The catheterization kit of claim 9, wherein the first and second tearable portions are detachable along any combination of a perforation, a guidance line, and a tear line.

11. The catheterization kit of claim 9, wherein the first tearable portion is a corner portion at a corner of the fluid collection bag, and the second tearable portion is a larger corner portion at the same corner of the fluid collection bag.

12. The catheterization kit of claim 9, further comprising an attaching member coupled to a top of the fluid collection bag, wherein the attaching member attaches to a seat in which a user is sitting during catheterization.

13. The catheterization kit of claim 9, further comprising a bubble stamped into the fluid collection bag, the bubble including an inlet and an outlet, the inlet being in fluid communication with the catheter, and the outlet being in fluid communication with a reservoir of the fluid collection bag, wherein a pressure of a fluid flowing through the bubble expands the bubble, and wherein a lack of pressure enables the bubble to deflate into a flattened state.

14. A method of manufacturing a fluid collection bag for a catheter, the method comprising:
welding a perimeter of two substantially rectangular sheets together to form a reservoir, wherein an inlet tube portion provides fluid communication between the reservoir and a catheter tube, and wherein a drain tube portion provides fluid communication between the reservoir and an external environment of the reservoir, said drain tube portion including a small-diameter portion and a large diameter portion;
sealing an end of the small-diameter portion;
etching a first tear line at a corner of the two rectangular sheets traversing the small-diameter portion of the drain tube to create a first tear-away portion; etching a second tear line at the same corner of the of the two rectangular sheets traversing the large-diameter portion of the drain tube to create a second tear-away portion; and
cutting a finger-sized hole in each tear-away portion to enable a user to detach the tear-away portion by pulling with a finger.

15. The method of claim 14, wherein etching the tear lines comprises etching a perforation along the tear lines.

16. The method of claim 14, wherein the welding the perimeter of the two substantially rectangular sheets comprises radiofrequency (RF) welding.

17. The method of claim 16, wherein etching the tear lines comprises RF welding along the tear lines.

18. The method of claim 14, wherein the substantially rectangular sheets are made of polyvinylchloride (PVC) of a thickness between 5 mils and 10 mils.
